(19) United States
(12) Patent Application Publication
(10) Pub. No.: US 2006/0121605 A1
(43) Pub. Date: Jun. 8, 2006

(54) Selection and propagation of progenitor cells

(75) Inventor: Nancy L. Parenteau, Benson, VT (US)

Correspondence Address: OLIFF & BERRIDGE, PLC, P.O. BOX 19928, ALEXANDRIA, VA, 22320, US

(73) Assignee: Organ Recovery Systems, Inc, Des Plaines (IL)

(21) Appl. No.: 10/559,474

(22) PCT Filed: Jun. 3, 2004
(86) PCT No.: PCT/US04/17284

§ 371 Date: Dec. 5, 2005

Related U.S. Application Data
(60) Provisional application No. 60/475,553, filed on Jun. 3, 2003.

(51) Int. Cl. A61K 35/12; C12N 5/06

(52) U.S. Cl. 435/325; 424/93.7

(57) ABSTRACT

The invention relates to a population of progenitor cells and methods for obtaining and culturing the progenitor cells. Methods and compositions of the present invention can be useful in fields including regenerative medicine (tissue regeneration), transplantation, and cancer research.

### CROSS-REFERENCE TO RELATED APPLICATION

The present application claims priority to and the benefit of U.S. Provisional Application Ser. No. 60/475,553, filed on Jun. 3, 2003, the entire disclosure of which is incorporated by reference herein.

### TECHNICAL FIELD

The invention generally relates to methods of progenitor cell selection, propagation and use. More particularly, the invention relates to methods and compositions for producing a population of progenitor cells in vitro.

### BACKGROUND OF THE INVENTION

Adult and embryonic stem cells are the subject of intense scientific interest because of their potential role in cell therapies. A potential stem cell source is the stem and progenitor cells that naturally reside in mature organs. However, the use of parenchymal progenitor cells has been hampered due to difficulties associated with their selective cultivation. For example, a major issue in the establishment of progenitor cell cultures from an adult pancreas or adult islet tissue is the overgrowth of contaminating non-parenchymal cell types and the continued presence of differentiation-committed cells.

Cultivation of islet progenitor cells is of particular interest as a potential treatment of insulin-dependent diabetes. Attempts have been made to cultivate islet cells derived from dissociated pancreatic tissue in serum-containing medium. However, the majority of serially propagated islet cell populations display only moderate proliferative capacity and retain differentiated properties. Fetal-derived progenitor bells, which are propagated with the aid of bovine brain extract, yield a cell population that gives rise to not only islet cells, but also acinar and ductal cells, and likely represents an earlier embryonic pancreatic progenitor as opposed to an islet precursor. Further, the method uses cells of embryonic origins which are naturally high in progenitor cell number, while it is more difficult to characterize and control progenitor cells in adult tissues. An islet cell population capable of producing insulin in vivo has been described. While the method allows for some degree of propagation of islet precursor cells, the cells require the concomitant co-propagation of stromal or “nurse” cells of a different tissue type such as the ductal cells, which represent the majority of the cells in the culture.

Alternative mechanical separation methods using, for example, cell markers, have been used to select for stem or progenitor cell populations. However, this artificial cell selection results only in a temporarily-enriched population of stem and progenitor cells.

None of the research has distinguished between the progenitor cells and their natural offspring, the transit amplifying cells, in the quest for obtaining a proliferating epithelial cell population containing a regenerative component. Hence, prior methods do not favor the maintenance of a progenitor cell pool over growth through transit amplification. Transit amplifying cells have a growth capacity that allows serial passages but they are naturally inhibitory to stem cell activation and continued expansion of progenitor cells (Hardin-Young et al. Current Neurovascular Research I, (2004); Parenteau, Encyclopedia of Animal and Plant Cell Technology, 365-78 (1999)). Failure to sustain progenitor cell activation and growth while controlling the generation and growth of transit amplifying cells or the survival of contaminating cell types has prevented the development and maintenance of substantially pure populations of adult progenitor cells. This difficulty has lead to variability experienced in the practice of human epithelial cell culture.

Thus, there exists a need for a method to produce a cell culture with the majority of the cells being parenchymal progenitor cells capable of prolonged expansion in vitro and organ regeneration with high fidelity in vivo. In addition, there exists a need for generating such cells from mature (adult and neonatal) tissue, especially parenchymal tissues.

### SUMMARY OF THE INVENTION

The present invention provides methods for selecting and expanding progenitor cell populations derived from neonatal or adult parenchymal tissue. Cultured populations of progenitor cells of the invention are a readily available source of cells which, when implanted in vivo, are useful to augment, repair, restore, or replace a diseased, damaged, missing, or otherwise compromised tissue or organ.

Methods of the invention provide culture conditions that promote selection of true progenitor cells. According to the invention, cell culture conditions are selected that undermine more differentiated cells, thus releasing the inhibitory influence that more differentiated cells normally have on the growth of progenitor cells. The result is a culture that allows the formation of colonies of self-supporting, undifferentiated progenitor cells that constitute a majority of the cell culture. The invention contemplates any serum-free culture conditions that induce a stress response in the cell culture to suppress the propagation of more differentiated cells yet permit progenitor cell growth. Ideally, conditions are selected so that once a population of progenitor cells has been created, tissue-specific differentiation can be induced, either in vitro or in vivo.

Although any set of culture conditions that promote progenitor cell growth are contemplated, a preferred method for propagating progenitor cells includes a serum-free medium that induces apoptosis or necrosis in the differentiating and/or differentiated cells. Cells may be initially cultured in a stringent primary medium with low or no level of either calcium and/or growth factors in order to bias the culture toward progenitor cell activation and growth. After progenitor cell growth has been initiated and the progentitor cells expand to be the majority of the cell population, the cells are propagated in a secondary, minimal growth medium that can be less stringent than the primary medium. Finally, differentiation of the resulting progenitor cell culture may be promoted by addition of differentiating factors in a tertiary medium in the presence of specific growth factors.

Alternatively, progenitor cells are harvested for use prior to differentiation. Any medium composition that inhibits growth of differentiated cells is contemplated as a means for generating a progenitor cell population according to the invention. Reducing the concentration and/or effectiveness of growth factors is one way to accomplish this goal. Inhibiting cell adhesion is another way. However, other methods, such as reducing the concentration of certain ions that normally promote growth of differentiated cells, inhibiting cell adhesion, changing culture pH, and others are. known in the art. Of course, a combination of any these individual techniques may be employed.

In a preferred embodiment, methods of the invention comprise providing in a serum-free medium a primary cell culture that includes a progenitor cell and at least one of a differentiating cell and a differentiated cell; inducing a stress response in the primary cell culture that permits the progenitor cell to replicate and suppresses propagation of the at least one of the differentiating cell and the differentiated cell; and identifying a population of progenitor cells resulting from the replication that constitutes a majority of cells in the primary cell culture. The methods may include steps of isolating progenitor cells from the primary cell culture and culturing the isolated progenitor cells to provide a secondary cell culture through no less than 5 serial passages. The secondary cell culture may be maintained in a defined culture medium including glutathione, e.g., between 0.01 to 10 mM glutathione. The methods may further include a step of stimulating differentiation of the population of progenitor cells.

In a preferred embodiment, serial passages are performed when the secondary cell culture is between about 60% to 75% confluent. The primary cell culture or the secondary cell culture may be maintained in the presence of a matrix component such as collagen.

A preferred stress response induces apoptosis and/or necrosis in the cell culture. And a preferred primary medium has substantially no growth factor or organ extracts, and no or a low level of calcium. Any cell type may be used to generate the primary culture, but epithelial cells are preferred, e.g., pancreatic cells, liver cells, and epidermal cells. A preferred method produces a cell population comprising at least about 60%, 70%, or 80% progenitor cells by number. Cells are cultured for a time sufficient to generate a population of progenitor cells.

Stimulation of differentiated cells is accomplished in the culture by changing culture conditions to bias toward formation of differentiated cells, such as by increasing differentiating factors.

The invention provides a substantially pure population of mammalian progenitor cells propagated in vitro from non-fetal tissue.

Additional methods of the invention comprise preventing or treating diabetes by culturing islet progenitor cells in vitro according to methods described above; and transplanting the progenitor cells into a mammal.

The foregoing, and other features and advantages of the invention, as well as the invention itself, will be more fully understood from the description and drawings that follow.

### BRIEF DESCRIPTION OF DRAWING

FIG. 1 illustrates cells related to parenchymal generation and a method of neogenesis using a progenitor cell pool.

The drawings are not necessarily to scale, emphasis instead generally being placed upon illustrating the principles of the invention. The advantages of the invention can be better understood by reference to the description taken in conjunction with the accompanying drawings.

### DETAILED DESCRIPTION OF THE INVENTION

The present invention provides, in part, methods for obtaining parenchymal progenitor cell population that is capable of self-sustained and prolonged expansion in vitro and organ regeneration in vivo and the resulting compositions. Methods comprise culturing primary cells in a culture medium that fails to support the maintenance of more differentiated cells, yet permits the growth and expansion of the underlying progenitor cell population. The result is a culture where progenitor cells constitute a majority, or preferably, a higher percentage, e.g., 60, 70, or 80 percent, of the cell population by number. In one embodiment, the cell culture is a substantially pure or homogenous population of progenitor cells. Once established, the progenitor cell population is propagated for multiple passages in defined conditions and, when desired, can be expanded for clinical treatment. One of the advantages of methods of the invention is that they provide a readily available source of progenitor cells that can be used in cell therapies.

Progenitor cells of the invention are derived from any organ or tissue containing parenchymal cells capable of regeneration including but not limited to, a cell population derived from pancreas, liver, gut, heart, kidney, cornea, skin, retina, inner ear, skeletal muscle, brain, or glands. In a preferred embodiment, the population of progenitor cells gives rise to cells of a specific parenchymal lineage, e.g. pancreatic islet endocrine lineage, liver hepatocyte cell lineage, or epidermal cell lineage.

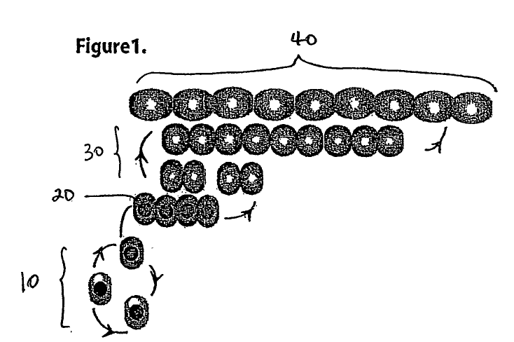

Referring to FIG. 1, to achieve neogenesis, i.e., de novo generation of functional tissue, methods of the present invention focus on the propagation and activation of a progenitor cell population in vitro. In one embodiment, a primary cell culture derived from an organ, e.g., the pancreas, contains multiple cell types. Resident stem cells 10 are slow-cycling cells that give rise to progenitor cells 20. Progenitor cells 20 are minimally differentiated cells and make up the proliferating cell compartment responsible for organ regeneration. The stem cells 10, which are slow-cycling, are distinct from the progenitor cell compartment and the transit amplifying cells 30 based on developmental studies, gene expression and apparent regulation by transcription factors. The progenitor cells 20, once activated, generate transit amplifying cells 30, which, in turn, lead to parenchymal cells 40. The transit amplifying cells 30 are lineage committed, differentiating cells, and exhibit limited replication. The parenchymal cells 40 are maximally differentiated functional cells.

According to one aspect of the present invention, a substantially pure or homogeneous population of progenitor cells can be cultivated outside the body, i.e., in vitro, without relying on cells from non-parenchymal tissues such as stromal, connective, or support tissues. In other words, the progenitor cells of the present invention are able to achieve self-sustained propagation from cells of its own tissue type. According to another aspect of the present invention, such a population of progenitor cells can be selected by controlling the condition of the cell culture to eliminate or at least inhibit more differentiated cells, including differentiating cells and differentiated cells. An advantage of such a methodology is that a progenitor is identified more by its behavior and the outcome of such behavior than by any marker it might express at any given time or location. Another advantage is that known mechanisms for regulating cell cycles, including those pertaining to apoptosis and necrosis, can be used to achieve the goal of the invention.

In a preferred embodiment, a population of substantially pure epithelial progenitor cells is produced in vitro by culturing a primary cell culture of epithelial cells in a primary culture medium that induces a stress response in the cells which depletes mature, differentiated parenchymal cells and/or differentiating transit amplifying cells. This response alters the dynamics of cell signaling in the culture to permit the progenitor cells to replicate and propagate. The stress response kills the more differentiated cells such that the resulting cell population is substantially free of differentiated or differentiating cells, and contaminating cells from other tissue types, e.g., stromal, fibroblast cells. While it is not yet certain, suppression of more differentiated cells may silence cell-to-cell signaling that inhibits the replication of progenitor cells and/or possibly provide signaling to activate progenitor proliferation. As a result, the progenitor cells propagate without any type of “feeder” or “nurse” cells from other tissue types.

There are various other ways to monitor the stress response besides visual observation. For example, the expression of a heat shock protein or an acute phase reactant gene can be measured as an indicator of the stress response.

One way to identify a pre-confluent colony of progenitor cells is to determine whether 5 the primary culture cells are undergoing active mitosis. Other ways include observing the cells under the microscope; or adding 5-bromo-deoxyuridine (BrdU), a thynidine analog, to the cell culture and detecting the incorporation of the BrdU into the cells using a monoclonal anti-BrdU antibody.

After a pre-confluent colony of progenitor cells is identified in the primary culture, it can be separated and used to establish a secondary cell culture comprising substantially homogeneous progenitor cells. The secondary cell culture may use the same type of culture medium as the primary culture, or use a medium that is less stringent. The secondary cell culture maintains the progenitor cells through multiple passages, and the progenitor cells retain the ability to differentiate or undergo neogenesis. In one embodiment, the progenitor cells undergo no less than five passages.

The present invention may further include steps to activate the progenitor cells to become differentiating cells, e.g., transit amplifying cells, and/or differentiated cells, e.g., parenchymal cells. The progenitor cells and/or their differentiating and/or differentiated offspring may be used in therapeutic applications, e.g., by implantation.

Throughout the description, where compositions are described as having, including, or comprising specific components, or where processes are described as having, including, or comprising specific process steps, it is contemplated that compositions of the present invention also consist essentially or, or consist of, the recited components, and that the processes of the present invention also consist essentially of, or consist of, the recited processing steps.

It should be understood that the order of steps or order for performing certain actions is immaterial so long as the invention remains operable. Moreover, two or more steps or actions may be conducted simultaneously.

Primary Cell Culture

The primary cell culture is designed to induce a stress response in differentiating and differentiated cells including contaminating cells of other tissue types, but to permit progenitor cells to propagate. It is believed that once the differentiating and differentiated cell population becomes depleted, the progenitor cells become activated, enter the cell cycle and start dividing with increasing rapidity. The stress response that initiates this selection process for the progenitor cells may be induced through a variety of means, for example, by inducing the apoptosis and/or necrosis of these cells. In one embodiment, the primary culture medium is chemically defined. “Chemically defined” means that the culture medium essentially contains no or substantially no serum or organ extracts. In certain embodiments, the medium contains a low level or is substantially free of growth factors. If a growth factor is present, it is preferably less than about 10 ng/ml, more preferably less than about 5 ng/ml, e.g., about 1 ng/ml. In one embodiment, the medium contains cAMP elevating agents, such as cholera toxin and foreskolin, preferably at a concentration of 9 ng/ml) to support the activation and outgrowth of the progenitor cells.

The primary culture medium may be designed to inhibit cell-cell adhesion. For example, the medium may contain nitric oxide which is known to inhibit cell adhesion and to disrupt cell-matrix interaction. Alternatively or in addition, tumor necrosis factor-alpha (TNF-α), interleukin 1-beta (ILl-β), and interferon-gamma (IFN-γ) can be added to stimulate nitric oxide-induced apoptosis. The cells also may be cultured in diluted hydrocolloid, dextran, and the like, to disrupt cell adhesion and to disfavor the survival of more differentiated cells.

In some embodiments, the medium contains a low level of or no calcium. If calcium is present in the culture medium, the concentration of calcium is preferably less than about 1 mM, e.g., between about 0.001 to about 0.9 mM. In one example, the calcium concentration is between about 0.01 to about 0.5 mM, and in another example, at about 0.08 mM. While not wishing to be bound by theory, the low calcium environment is thought to limit the cell-to-cell contact that is necessary for the interaction and maintenance of the more differentiated cells. A low calcium environment combined with the chemically-defined culture medium and minimal concentration of growth factors causes the differentiated cells to divide more slowly, eventually causing those cells to undergo apoptosis resulting in a population of progenitor cells within the culture.

Many apoptosis or necrosis-related pathways are known in the art. The primary culture medium can be designed to initiate or enhance such pathways. Pathways that down-regulate such stresses can be deactivated—for example, protein kinases that inhibit apoptosis can be blocked. Many of these pathways are cooperative and can be used in combinations. Examples of such signaling pathways include the caspase pathways, the Bcl-2 pathways, and the interleukin-10 pathway.

The caspase pathway involves nuclear factor kappa B (NF-κB) which is a transcription factor that, once translocated to the nucleus, activates transcription of various genes including those affecting the onset of cell death. Ligands, antigens, antibodies, growth factors, cytokines, lymphokines, chemokines, cofactors, hormone and other factors that regulate NF-κB, such as tumor necrosis factor (TNF) can be added to the culture medium to kill the differentiating and/or differentiated cells through the caspase, Bcl-2, and other pathways. Such factors include TNF-α, TNF-like weak inducers of apoptosis (TWEAK), TNF-related apoptosis-inducing ligands (TRAIL), interleukins (IL) (e.g., IL 10), Fas ligands, Apoptosis inducing protein ligands (e.g., APO-3L and 2L), transforming growth factor beta (TGF-β), endotoxins (e.g., lipopolysaccharide), regulated-upon-activation normal T-cell expressed and secreted (RANTES), interferons (e.g., IFN-γ), oxadaic acid (a serene/threonine protein-phosphatase inhibitor) and so on.

Examples of apoptosis-inhibiting signaling pathways that can be disrupted to disfavor the survival of more differentiated cells include the AKT-mediated signaling pathway and those activated by other so-called “survival kinases” such as IKK, erk, Raf-1. Possible ways to interfere with the AKT signaling pathway include use of siRNA, growth factors such as those produced by autocrine/paracrine, and/or antibodies to block the receptor tyrosine kinase AKT. Alternatively, elimination of growth factors that could induce this pathway using a stringent, defined medium may lead to similar results. Another example of disrupting apoptosis-inhibiting signals includes deactivation of heat shock protein 70.

Other environmental factors' such as heat, radiation, humidity, and pH also can lead the desired stress response in the cell culture. For example, ultraviolet radiation may induce cell death in more differentiated cells.

Secondary Cell Culture

The secondary cell culture typically comprises progenitor cells selected from the primary cell culture by virtue of their ability to thrive in the stressed conditions. The secondary cell culture maintains the progenitor cells so that they maintain the potential to differentiate or under neogenesis without actual differentiation. The ability of a population of progenitor cells to endure prolonged propagation through serial passage brings about another advantage of the present invention, which is to ability to amass a sufficient amount of progenitor cells for neogenesis and other applications.

The secondary cell culture may use the same type of medium as the primary culture as it continues to suppress differentiation of the progenitor cells. Alternatively, the secondary culture medium may be less stringent. Some limited amount of growth factors may be added to the base medium since the culture initially is substantial free of more differentiated cells. Some non-essential growth factors can be used sparingly or intermittently in the secondary culture medium. Examples of such growth factors include epidermal growth factor (EGF), transforming growth factor alpha (TGF-α), keratinocyte growth factor (KGF) and basic fibroblast growth factor.

Further Regulation

Cells harvested from a primary or secondary culture can be further regulated. In certain embodiments, progenitor cells are induced to differentiate progressively into various stages as described earlier with reference to FIG. 1. A tertiary medium may be prepared with differentiating factors such as a higher level of calcium, serum and/or TGF-β. The medium may also include dexamethasone and cyclic adenosine monophosphate (cAMP) elevating agents, and other factors known to promote and sustain the growth of differentiating cells. Cell differentiation may also be promoted by addition of extracellular matrix, hydrogel or hydrocolloid substances or polymers that can assist the formation of cellular complexes. Such cells are applied in various therapies.

### EXAMPLES

The following examples are provided to illustrate the principle of the present invention and should not be interpreted in any way as limiting the scope of the claims. Those skilled in the art will recognize that various modifications can be made without departing from the spirit and scope of the present invention.

### Example 1

### Culturing Conditions

Progenitor cells derived from human tissue are established by enzymatically dissociating the tissue of interest or mincing to form 1-2 mm² tissue explants. If enzymatic digestion is used, enzymes such as collagenase, hyaluronidase, dispase, pronase, trypsin, elastase and chymotrypsin are preferred. Numerous methods of preparing a primary cell culture are known in the art.

Cultures are initiated by flattening and spreading a heterogeneous cell population onto a tissue culture substrate, such as a plate coated with Type I collagen. Typically, the majority of cells exhibit a large, spread epitheliod to fibroblastic appearance. The cells are then cultured in a chemically-defined culture medium that contains little or no calcium and very little or no growth factors. By chemically-defined conditions it is meant that the culture medium contains essentially no serum or organ extracts. If calcium is present in the culture medium, the concentration of calcium is preferably less than about 1 mM, e.g., between about 0.001 to about 0.9 mM. In another example, the calcium concentration is about 0.08 mM. If growth factor is present, its concentration is less than about 10 ng/ml, and preferably less than about 5 ng/ml, e.g., at about 1 ng/ml.

Single parenchymal progenitor cells and colonies of parenchymal progenitor cells are identified within the first 10 days. Usually, the colonies are visually distinct from other cells. Unlike most cells, the parenchymal progenitor cells remain small, rounded or hexagonal in shape. The progenitor cells are typically less than about 15 microns and have a dense appearance. Those cells are refractory and are readily-identified using phase-contrast microscopy. Moreover, the parenchymal progenitor cells can be identified by their active mitosis. Typically, colonies of parenchymal progenitor cells increase in number to become the predominant population in the primary culture within about 14 days. The cells are harvested by trypsinization when the loosely formed colonies and small dividing cells occupy about 50-70% of the cell culture surface. In one embodiment, the progenitor cells occupy about 80% of the cell culture surface.

The resulting progenitor population in a secondary culture is characterized as having a small size, a plating efficiency of about 40% or greater upon passage, and rapid cell division of about 36 hours or less. The progenitor cells are passaged for at least about 5 passages and can extend to about 13 passages, or more, depending on the split ratios used during passage. The cells typically achieve about 10 population doublings or greater. Cells maintain characteristics of tissue-specific progenitor cells, such as expression of lineage specific genes and genes developmentally associated with progenitor cells.

The progenitor cells have the ability to exhibit organotypic differentiation upon changing the culture conditions to an environment, i.e., a tertiary culture medium, that may contain factors that promote and/or support development and growth of differentiating cells. Examples of such factors include hydrocortisone, TGF-β, hepatocyte growth factor, or other factors that have been identified as effective in regulating embryonic organogenesis. Examples of other environmental conditions that can be introduced to the tertiary culture include the addition of an extracellular matrix to promote cluster formation or three-dimensional culture, the addition of calcium at a concentration greater than about 1.0 mM, or any method which allows cell-cell adhesion to occur and tissue architecture to develop. Any of these factors and conditions may be used together or in sequence to advance organogenesis depending on the tissue type.

The selection of a population of pancreatic islet progenitor cells is described below. However, that example is not intended to be limiting and progenitor cells can be derived from any organ or tissue containing parenchymal cells capable of regeneration such as the liver, gut, heart, cornea, skin, retina, inner ear, skeletal muscle, brain, or glands.

### Example 2

### Pancreatic Islet Cells

The endocrine progenitor cells are derived from either whole neonatal pancreas or isolated adult pancreatic islets. The cells are then cultured under stringent conditions to impose a stress condition on the cell culture in order to select for growth of an endocrine progenitor cell population. Once established, this population is propagated for multiple passages undifferentiated and thereby expanded for clinical treatment of insulin dependent diabetes.

The stress-inducing culture medium of the invention allows for the establishment of primary cultures and facilitates the identification of a subpopulation of cells from these primary cultures that can then be serially passaged, thus providing for an expanded number of cells that could have therapeutic value. Preferably, the stress-inducing culture medium consists of a chemically defined medium without serum or growth factors. Cells grown from the pancreatic or islet tissue using this medium and culture methodology show a predominantly epithelial-like morphology and express cytokeratin markers characteristic of epithelial cells.

As the cells are expanded in culture, they are characterized by expression of markers associated with pancreatic progenitor cells, such as PDX 1. The homeodomain protein PDX1 is required at an early stage in pancreas development (Nature Genetics 15:106-110 (1997); Development 122:1409-1416 (1996)). As differentiated endocrine cells appear, they separate from the epithelium and migrate into the adjacent mesenchyme where they cluster. PDX1 is later required for maintaining the hormone-producing phenotype of the β-cell by positively regulating insulin and islet amyloid polypeptide expression and repressing glucagon expression (Genes Dev 12:1763-1768 (1998)). PDX-1 is also required to regulate GLUT2 expression in β-cells suggesting an important role in maintaining normal β-cell homeostasis.

Neurogenin-3, a member of the mammalian neurogenin gene family, has been established as a proendocrine gene (See Proc Natl Acad Sci USA 97: 1607-11 (2000); Curr Opin Genet Dev 9:295-300 (1999)) and is considered a marker of islet progenitor cells during development (Development 129: 2447-57 (2002)). The progenitor cell characteristic of the islet-derived cell population expresses neurogenin-3.

The endocrine progenitor cells may be induced to differentiate using chemical or physical means, such as by supplementing the culture medium with an agent that promotes differentiation to insulin-producing beta cells or by inducing morphological changes such as cell cluster formation in the presence of extracellular matrix. The cells may also be induced to differentiate as a result of implantation into a permissive environment. For example, in vivo differentiation may be seen upon implantation under the kidney capsule, subcutaneously, or in the submucosal space of the small intestine.

### Example 3

### Culture Medium

A stringent, stress-inducing culture medium used for the primary culture contains no or essentially no serum or organ extracts.

A primary culture medium of the invention is provided with a nutrient base, which may or may not be further supplemented with other components. The nutrient base may include inorganic salts, glucose, amino acids and vitamins, and other basic media components. Examples include Dulbecco's Modified Eagle's Medium (DMEM); Minimal Essential Medium (MEM); M199; RPMI 1640; Iscove's Modified Dulbecco's Medium (EDMEM); Ham's F12, Ham's F-10, NCTC 109 and NCTC 135. A preferred base medium of the invention includes a nutrient base of either calcium-free or low calcium DMEM without glucose, magnesium or sodium pyruvate and with L-glutamine at 4.0 mM, and Ham's F-12 with 5 mM glucose in a 3-to-1 ratio. The final glucose concentration of the base is adjusted to preferably about 5 mM. The base medium is supplemented with one or more of the following components known to the skilled artisan in animal cell culture: insulin or an insulin-like growth factor; transferrin or ferrous ion; triiodothyronine or thyroxin; ethanolamine and/or o-phosphoryl-ethanolamine, strontium chloride, sodium pyruvate, selenium, non-essential amino acids, a protease inhibitor (e.g., aprotinin or soybean trypsin inhibitor (SBTI)) and glucose.

In one example, no growth factor is added to the medium. In another example, the base medium is further supplemented with components such as non-essential amino acids, growth factors and hormones. For example, TGF-β is added as an a poptogen for promoting apoptosis of differentiated liver cells or TNFα is added as an a poptogen of differentiated islet, liver and epidermal cells. Defined culture media which can be useful in the present invention are described in U.S. Pat. No. 5,712,163 to Parenteau and is incorporated herein by reference. Titration experiments can be used to determine the appropriate concentrations for the supplements, as known by one skilled in the art. Examples of preferred concentrations are provided as follows:

A preferred concentration of insulin in the secondary medium is 5.0 μg/ml. Proinsulin, insulin-like growth factors such as IGF-1 or II may be substituted for insulin. Insulin-like growth factor as used herein means compositions which are structurally similar to insulin and stimulate the insulin-like growth factor receptors.

Preferably, ferrous ion is supplied by transferrin in the secondary medium at a concentration of from about 0.05 to about 50 μg/ml, a preferred concentration being about 5 μg/ml.

Triiodothyronine is added to maintain rates of cell metabolism. It is preferably present at a concentration of from about 2 to about 200 pM, more preferably at about 20 pM.

Either or both ethanolamine and o-phosphoryl-ethanolamine may be used in the practice of the present invention. Both are phospholipids that function as precursors in the inositol pathway and in fatty acid metabolism. Supplementation of lipids that are normally found in serum may be necessary in a serum-free medium. Either or both ethanolamine and o-phosphoryl-ethanolamine are provided to the media at a concentration range of preferably between about 10⁻⁶ M to about 10⁻² M, more preferably between about 10⁻⁴ M.

Selenium may be used at a concentration between about 10⁻⁹ M to about 10⁻⁷ M, preferably at about 5×10⁻⁸ M. And amino acid L-glutamine or its substitute may be used at a concentration between about 1 mM to about 10 mM, preferably at about 6 mM.

When preparing the secondary medium for serial passage of progenitor cells, other components may be added to the media, depending upon, e.g., the particular cell being cultured, including but not limited to, epidermal growth factor (EGF), transforming growth factor alpha (INF-α), keratinocyte growth factor (KGF), and basic fibroblast growth factor (bFGF). EGF as an optional component in a secondary medium may be used at a concentration as low as 1 ng/ml.

A preferred embodiment of the secondary medium includes: a base 3:1 mixture of DMEM (no glucose, no calcium and 4 mM L-glutamine) and Ham's F-12 medium supplemented with the following components to achieve the final concentration indicated for each component: 6 mM L-glutamine (or equivalent), 1 ng/ml EGF, 1×10⁻⁴ M ethanolamine, 1×10⁻⁴ M o-phosphorylethanolamine, 5 μg/ml insulin, 5 μg/ml transferrin, 20 pm triiodothyronine, 6.78 ng/ml selenium, 24.4 μg/ml adenine, 1 mM strontium chloride, 100 mM sodium pyruvate, 10 mM non-essential amino acids, and 5 mM glucose.

While the cell population propagated according to the invention comprises a pool of progenitor cells at one stage, further commitment to differentiation and organ development may be induced. A tertiary medium allows the progenitor cells to generate a majority of transit amplifying cells and advance organogenesis when desired. A preferred embodiment of the medium for the generation of transit amplifying cells includes a basem mixture of 1:1 DMEM (no glucose, no calcium and 4 mM L-glutamine) and Ham's F-12 medium supplemented with the following components to achieve the final concentration indicated for each component: 6 mM L-glutamine (or equivalent), 10 ng/ml EGF or HGF or both (depending on cell type), 1×10⁻⁴ M ethanolamine, 1×10⁻⁴ M o-phosphorylethanolamine, 5 μg/ml insulin, 5 μg/ml transferrin, 20 pm triiodothyronine, 6.78 ng/ml selenium, 24.4 μg/ml adenine, 100 mM sodium pyruvate, 2×10⁻⁹ progesterone, 1.1 μM hydrocortisone, 0.08 mM calcium chloride and 9 ng/mL forskolin.

Progenitor cells may be plated at a moderate density of between 1,000 to 5,000 cells per cm² in this medium on a collagen-coated plastic surface and cultured for at least one passage. The cell population may be used as is or further differentiation may be initiated. Where further differentiation is desired, the cells are transferred to conditions where forskolin is removed from the tertiary medium and the calcium concentration is increased, e.g., to about 1.88 mM. Other changes to the culture environment also may be included at this time, e.g., addition of an extracellular matrix component. Some of the changes in the environmental condition depend on the tissue type, e.g. epidermal cells may be cultured at an air-liquid interface, islet cells may be cultured in a matrix condition that promotes cluster formation, and hepatocyte cells may be cultured in a 3-dimensional substrate that promotes cord formation.

A typical way of preparing media useful for the present invention is set forth below. However, components of the present invention may be prepared using other conventional methodology with or without substitution in certain components with an analogue or functional equivalent. Also, concentrations for the supplements may be optimized for cells derived from different species and cell lines from different organisms due to factors such as age, size and health. Titration experiments can be performed with varying concentrations of a component to arrive at the optimal concentration for that component.

The medium, whether primary, secondary or tertiary, is prepared under sterile conditions, starting with base medium and components that are bought or rendered sterile through conventional procedures, such as filtration. Proper aseptic procedures are used throughout the Examples. DMEM and F-12 are combined and the individual components are then added to complete the medium. Stock solutions of all components can be stored at −20° C., with the exception of the nutrient source that can be stored at 4° C.

A vessel suitable for animal cell or tissue culture, e.g., a culture dish, flask, or roller bottle, is used to culture the endocrine progenitor cells. Materials such as glass, stainless steel, polymers, silicon substrates, including fused silica or polysilicon, and other biologically compatible materials may be used as cell growth surfaces. The cells of the invention may be grown on a solid surface or a porous surface, such as a porous membrane, that would allow bilateral contact of the medium to the cultured cells. In addition, the cell growth surface material may be chemically treated or modified, electrostatically charged, or coated with biological agents such as with peptides or matrix components. The preferred growth surface for carrying out the invention is a conventional tissue culture surface coated with Type I collagen.

The cultures are preferably maintained between about 3420 C. to about 38° C., more preferably 37° C., with an atmosphere between about 5-10% CO₂ and a relative humidity between about 80 to 90%. An incubator is used to sustain environmental conditions of controlled temperature, humidity, and gas mixture for the culture of cells.

Medium used during the first step in progenitor cell activation from a resident progenitor cell can be harvested and used to promote activation of progenitor cells still residing in the expanded culture. Similarly, conditioned medium from proliferating later passage cells can be used to support proliferation of progenitor cells plated at low density. The conditioned medium can comprise from 10-50% of the nutrient medium. Alternatively, a more specialized conditioned supplement is created by removing the common heparin-binding growth factors, concentrating, and desalting the harvested medium. The concentrated supplement can be used at a concentration equivalent to the original starting material. More detailed examples are provided in Example 7 below.

### Example 4

### Transplantation

The invention provides for methods of transplantation into a mammal. A progenitor cell as described above can be transplanted or introduced into a mammal or a patient. In one example, transplantation involves transferring a progenitor cell into a mammal or a patient by injection of a cell suspension into the mammal or patient, surgical implantation of a cell mass into a tissue or organ of the mammal or patient, or perfusion of a tissue or organ with a cell suspension. The route of transferring the progenitor cell or transplantation will be determined by the need for the cell to reside in a particular tissue or organ and by the ability of the cell to find and be retained by the desired target tissue or organ. In the case in which a transplanted cell is to reside in a particular location, it can be surgically placed into a tissue or organ, e.g., the duodenum, or injected into the bloodstream or related organ if the cell has the capability to migrate to the desired target organ as is the case with liver cells which can locate to the liver when injected into the portal circulation or spleen.

The invention specifically contemplates transplanting into patients isogeneic, allogeneic, or xenogeneic progenitor cells, or any combination thereof.

### EXAMPLE 5

### Treating Insulin-Dependent Diabetes Using Pancreatic Progenitor Cells

Progenitor cells are useful to replace lost beta cells from Type 1 diabetes patients or to increase the overall numbers of beta cells in Type 2 insulin-dependent diabetic patients. Cadaveric tissue preferably serves as the donor tissue used to produce progenitor cells. Islets are isolated from the tissue and progenitor cells are selected as described herein. The progenitor cells can be transplanted into the patient directly following culture expansion or after a period of differentiation which may be induced by growth factors, hormones and calcium. In one embodiment, the progenitor cells are immunologically tolerated, such that in allogenic transplants, they do not illicit a humoral or immune cell response. In one aspect of this embodiment of the invention, these cells do not normally express MHC class II antigens and do not elicit a costimulatory response that initiates T cell activation.

In another embodiment of the invention, the recipient of the transplant may demonstrate an immune response to the transplanted cells which can be combated by the administration of blocking antibodies to, for example, an auto antigen such as GAD65, by the administration of one or more immunosuppressive drugs described herein, or by any method known in the art to prevent or ameliorate alloimmune and/or autoimmune rejection.

### Example 6

### Drug Discovery

The unique properties of a population of adult organ progenitor cells, especially a concentrated or substantially pure population, make those cells a highly suitable and desirable tool for characterizing organ regulation and mechanisms of autocrine growth regulation, for example. This is particularly relevant to carcinogenesis and study of how to stimulate in vivo regeneration. The fact that human cells can be used is particularly beneficial. The ability to use the system under chemically defined conditions is also advantageous for research and analysis.

In some embodiments, the cell population cultured according to the invention is characterized using gene chip analysis, polymerase chain reaction, and/or proteomics analysis at various stages in the method described hereinabove: primary activation from the mature organ, secondary growth and serial passages, and under tertiary conditions promoting differentiation. By comparing the genes activated and proteins produced, and their level of expression at each stage using the same cell strain, differences can be observed that directly relate to changes in regulation of the cell population. These responses can then be compared in more than one human cell strain derived from like or different organs under varying conditions to arrive at common cellular pathways governing human cell populations in the adult organ. These pathways then become candidate targets for biopharmaceutical or pharmaceutical manipulation. Once targets are identified, compounds may be tested in the system to confirm their role in the regulation of the human cells or organotypic tissues.

### Example 7

### Establishment and Use of a Progenitor Cell Population from Isolated Human Islets of Langerhans

Isolation of human islets is performed using the semi-automated method originally proposed by Ricordi (Diabetes 37:413-420, 1988). Procured organs are distended by intraductal infusion of Liberase HI (Roche Molecular Biosciences, Indianapolis Ind.) or Serva collagenase (Crescent Chemical, Brooklyn N.Y.). After a process of continuous digestion for approximately 12 to 30 min, tissue is collected into about 8 liters of Hanks solution and washed. Free islets are separated from the other tissue using a continuous gradient of EuroFicoll in a Cobe 2991 cell separator (Cell Tiss Res. 310:51-58, 2002).

About 200 islet equivalents are plated into 60-mm collagen-coated culture dishes containing 4 ml of primary medium consisting of the 3:1 base of DMEM (no glucose, no calcium, with 4 mM L-glutamine) and Ham's F12 supplemented with the following components with the final concentration of each component indicated: 6 mM L-glutamine (or equivalent), 1×10⁻⁴ M ethanolamine, 1×10⁻⁴ M o-phosphoryl-ethanolamine, 5 μg/ml insulin, 5 μg/ml transferrin, 20 pM triiodothyronine, 6.78 ng/ml selenium, 24.4 μg /ml adenine, 1 mM strontium chloride, 1 mM sodium pyruvate, 100 μM non-essential amino acids, 25 μg/ml aprotinin, 9 ng/ml forskolin and 5 mM glucose.

Cultures are incubated for 14 days during which time cells spread from the isolated islets. The progenitor small cell population that emerged is harvested by trypsinization at 70% confluence.

Serial Passage of Islet-Derived Progenitor Cells.

Proliferating progenitor cells are serially passaged at 4000 cells per cm² on Type I collagen coated dishes in a secondary medium consisting of 3:1 DMEM (no glucose, no calcium, with 4 mM L-glutamine): F12 base medium supplemented with the following components with the final concentration of each component indicated: 6 mM L-glutamine (or equivalent), 1 ng/ml epidermal growth factor, 1×10⁻⁴ M ethanolamine, 1×10⁻⁴ M o-phosphoryl-ethanolamine, 5 μg/ml insulin, 5 μg/ml trannsferrin, 20 pM triiodothyronine, 6.78 ng/ml selenium, 24.4 μg/ml adenine, 1 mM strontium chloride, 1 mM sodium pyruvate, 100 μM non-essential amino acids, and 5 mM glucose. Epidermal growth factor is added at feeding when the cells have established and reached at least 30% confluence. Cells are passaged at 80% confluence or less.

Heparin Fractionated Conditioned Medium for Selective Stimulation of Progenitor Cells.

Conditioned medium is harvested from activated proliferating progenitor cell cultures and passed over a preparative heparin-sepharose to remove heparin binding growth factors. The void fraction is concentrated by filtration and desalted using a G-100 sepharose column. The concentrated fraction is filter sterilized, aliquoted and stored at −70° C. until use. The concentrated fraction is reconstituted to its original volume with fresh supplemented base medium and used to support the propagation of late passage or low density progenitor cells.

Conditioned Medium for the Activation of Progenitor Cells

Cultures are established from islets as described above. Conditioned medium is harvested from cultures at the intermediate stage during apoptosis of differentiated cells and the beginning of progenitor colony formation. The conditioned medium is concentrated by filtration and desalted using a G-100 sepharose column. The concentrated fraction is reconstituted to its original volume with fresh supplemented base medium and used to support the activation of new progenitor cells derived using cell sorting or other methods such as culture methods to produce cultures of slow-cycling pancreatic small cells.

In vivo Differentiation of Islet Progenitor Cells

Islet progenitor cells are serially cultivated to passage 6. The typsinized cells are suspended in base medium and delivered laproscopically via a large needle into the submucosal space of the duodenum. The progenitor cells cluster and differentiate into insulin-producing islet tissue.

In vivo Delivery of Partially Differentiated Islet Progenitor Tissue

Islet progenitor cells are serially cultivated to passage 6 and trypsinized. The cells are plated onto tissue culture plastic in the presence of the supplemented basal medium described above with the addition of 1.8 mM calcium chloride, 10 ng/mL forskolin hydrocortisone at 4 μg/ml and an overlay of Type I collagen. Cystic structures form. The cysts may be harvested and delivered as is or treated to undergo further differentiation by the removal of the forskolin and collagenase treatment to remove the collagen overlay. Alternatively, the cell suspension is inoculated in a zero gravity culture system which promotes the formation of suspended cell clusters in the presence of the supplemented basal medium described above with the addition of 1.8 mM calcium chloride and hydrocortisone at 4 μg/ml. The cysts or partially differentiated clusters are injected laporoscopically using a trochar into the submucosal space of the duodenum or alternatively into the portal vein of the liver.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

Each of the patent documents and scientific publications disclosed hereinabove is incorporated by reference herein for all purposes.

We claim:

1. A method of propagating a progenitor cell in vitro, the method comprising the steps of:
providing in a serum-free medium a primary cell culture comprising a progenitor cell and at least one of a differentiating cell and a differentiated cell;
inducing a stress response in the primary cell culture, wherein the stress response permits the progenitor cell to replicate and suppresses propagation of the at least one of the differentiating cell and the differentiated cell; and
identifying a population of progenitor cells resulting from replication, wherein the population of progenitor cells constitutes a majority of cells in the primary cell culture.

2. The method of claim 1 wherein the stress response comprises apoptosis.

3. The method of claim 1 wherein the stress response comprises necrosis.

4. The method of claim 1 comprising the step of isolating progenitor cells from the primary cell culture.

5. The method of claim 4 comprising the step of culturing the progenitor cells to provide a secondary cell culture.

6. The method of claim 5 wherein the culturing step comprises no less than 5 passages of the progenitor cell population.

7. The method of claim 1 wherein the primary cell culture comprises cells selected from the group consisting of epithelial cells, pancreatic cells, and liver cells.

8. The method of claim 7 wherein the at least one of the differentiating cell and the differentiated cell is a ductal epithelial cell, a nurse cell, a stromal cell, or a fibroblast cell.

9. The method of claim 1 wherein the medium comprises substantially no organ extracts.

10. The method of claim 1 wherein the medium comprises between about 0 mM to about 0.9 mM calcium ion.

11. The method of claim 10 wherein the medium comprises calcium ion at a concentration about 0.08 mM.

12. The method of claim 1 wherein the medium comprises substantially no growth factor.

13. The method of claim 1 wherein the medium is designed to inhibit cell adhesion.

14. The method of claim 1 wherein the medium comprises at least one of a ligand, an antigen, an antibody, a growth factor, a cytokine, a lymphokine, a chemokine, a cofactor, and a hormone.

15. The method of claim 1 wherein the step of inducing the stress response comprising regulating at least one of a caspase pathway, a Bcl-2 pathway, an interleukin-10 pathway, and an AKT-mediated pathway.

16. The method of claim 1 wherein the medium comprises at least one of a tumor necrosis factor (TNF), a TNF-like weak inducer of apoptosis (TWEAK), a TNF-related apoptosis-inducing ligand (TRAIL), an interleukin (IL), a Fas ligand, an Apoptosis inducing protein ligand, a transforming growth factor, an endotoxin, a regulated-upon-activation normal T-cell expressed and secreted (RANTES) molecule, an interferon (IFN), and an oxadaic acid.

17. The method of claim 1 wherein the medium comprises at least one molecule selected from the group consisting of nitric oxide, TNF-α, IL-10, IL 1-β, APO-3L, APO-2L, TGF-β, IFN-γ, and lipopolysaccharide.

18. The method of claim 1 wherein the medium comprises an elevating agent of cyclic adenosine monophosphate (cAMP).

19. The method of claim 1 wherein the population of progenitor cells consists of at least about 80% progenitor cells by number.

20. The method of claim 1 further comprising the step of stimulating differentiation of the population of progenitor cells.

21. A method for preventing or treating a disease or condition in a mammal, the method comprising the step of:
transplanting into a mammal the progenitor cells isolated according to the method of claim 4.

22. The method of claim 21 wherein the disease is diabetes.

23. An in vitro progenitor cell population comprising progenitor cells maintained in a defined culture medium, wherein the defined culture medium induces a stress response in the cell culture and wherein the progenitor cell population constitutes a majority of all cells in the medium by number.

24. The in vitro progenitor cell population of claim 23 wherein the defined culture medium is free of both serum and growth factors and contains a calcium ion at a concentration of no more than about 0.09 mM, and wherein the progenitor cell population constitutes no less than 80% of all cells in the medium, and the progenitor cells are capable of differentiation and neogenesis.

25. A substantially pure population of mammalian progenitor cells propagated in vitro from non-fetal tissue.
